(19) United States
(12) Patent Application Publication
(10) Pub. No.: US 2005/0171605 A1
(43) Pub. Date: Aug. 4, 2005

(54) Cervical prosthesis and instrument set

(75) Inventors: Helmut D. Link, Hamburg (DE); Arnold Keller, Kayhude (DE); Paul C. McAfee, Baltimore, MD (US)

Correspondence Address: Barry E. Bretschneider;Morrison & Foerster LLP, Suite 300, 1650 Tysons Boulevard, McLean, VA, 22102, US

(73) Assignee: CERVITECH, Inc., Rockaway, NJ (US)

(21) Appl. No.: 10/768,713

(22) Filed: Feb. 2, 2004

(51) Int. Cl. A61F 2/44; A61B 17/16

(52) U.S. Cl. 623/17.11; 606/84

(57) ABSTRACT

Cervical prosthesis consisting of a lower cover plate (10) which is to be connected to a lower vertebral body, an upper cover plate (11) which is to be connected to an upper vertebral body, and a prosthesis core (12) which forms a hinged connection between the upper cover plate and lower cover plate. The bottom surface of the lower cover plate (10) is substantially flat. The top surface (14) of the upper cover plate (11) is convex at least in sagittal section.

The invention relates to a cervical prosthesis consisting of a lower cover plate, an upper cover plate, and a prosthesis core which forms a hinged connection between the upper cover plate and lower cover plate. The lower cover plate is to be connected to a lower vertebral body, and the upper cover plate is to be connected to an upper vertebral body.

It is known for the outer surfaces of the prosthesis cover plates to be designed flat (EP-A1-344508) or with a convex bulge (EP-A-1166725; WO-A-0211650). To ensure that these surfaces can cooperate properly with the vertebral body surfaces delimiting the intervertebral space, said vertebral body surfaces are milled to obtain the correct shape (WO-A-03075774; U.S. Pat. No. 6,159,214; WO-A-03063727). No consideration is given to preserving the compact and resistant, yet very thin, cortical bone of the end plates of the vertebral bodies. When using prostheses which are circular in the horizontal plane, the spatial extent of the vertebral end plates is not sufficiently utilized either, leading to unnecessarily high surface pressure.

The object of the invention is to make available a cervical intervertebral prosthesis which better exploits the natural circumstances and permits reduced bone removal. It is also an aim of the invention to make available a cervical intervertebral prosthesis which is held securely in the intervertebral space. The invention is based on the observation that the upwardly directed vertebral body end plate, which delimits the intervertebral space at the bottom, has a shape which approximates to a flat configuration, whereas the lower vertebral cover plate, which delimits the intervertebral space at the top, is concavely curved at least in the sagittal plane.

Based on these observations, the invention provides an intervertebral prosthesis in which the outer surface of the lower cover plate is substantially flat, while the outer surface of the upper cover plate has a convex bulge. If the vertebral surfaces are shaped according to the shape of the outer surfaces of the prosthesis, the greater similarity between the natural vertebral body surfaces on the one hand and the outer surfaces of the prosthesis on the other means that, compared to the previously known prostheses, only a fairly small amount of bone has to be removed.

The fact that the prosthesis surfaces are well adapted to the natural conditions results in a secure hold of the prosthesis. This applies in particular to the connection between the upper, convexly dome-shaped surface of the prosthesis and the associated vertebral surface. It goes without saying that, for the purpose of ensuring a secure hold of the prosthesis on the bone, other known means can also be used, for example providing a rough or porous surface form of the prosthesis and equipping the prosthesis surface with substances which promote bone growth and intimate contact between bone and prosthesis.

To permit the best possible adaptation to the natural bone shape, the top face of the upper cover plate of the prosthesis has a bulge which, in sagittal section, lies between a circle contour with a radius of curvature of not more than 25 mm and an acute-angled contour with an apex angle of not more than 90°, preferably 60°. This in many cases makes it possible for the physician, when shaping the interacting lower end plate of the upper vertebra, to preserve part of the cortical bone or of the more compact structure closely adjoining this inside the vertebra. This applies in particular to those areas lying radially farther to the outside when looking at the underside of the vertebral body in a plan view.

In an advantageous embodiment, the top face of the upper cover plate is formed by a surface of rotation. This has the advantage that the prosthesis can be produced inexpensively and that the operating procedure is made easier too, because the work involved in shaping the bone surface is facilitated. The set of instruments used for the shaping work is also simplified.

However, the top face of the upper cover plate of the prosthesis in the direction of the frontal plane can advantageously also be made elongate, because the cover plate of the upper vertebral body delimiting the intervertebral space at the top is also slightly elongate in this direction. The underside of the lower cover plate of the prosthesis can also be elongate in this direction, as is known per se.

It is particularly advantageous if the top face of the upper cover plate is made up of three surface portions, of which the two outer portions are opposite surfaces of half rotation, and of which the portion lying between them consists of parallel generatrices which connect the mutually facing boundary lines of the outer surface portions to one another. This affords the possibility of shaping the associated bone surface using a milling tool which is a body of rotation suitable for the production of the two outer surfaces of half rotation and also of the part lying between them.

Such an instrument for milling the vertebral surface intended to cooperate with the top face of the upper cover plate of the prosthesis can be distinguished by the fact that it has a base plate suitable for bearing on the lower vertebral body, and comprises a milling tool used for working the upper vertebral body and mounted on the base plate. The base plate can be configured such that its contour approximates to the surface shape of the lower vertebral body in order to facilitate centering of the instrument in the intervertebral space.

The milling tool can have an axis of rotation arranged transversely with respect to the base plate. It will then cut out, from the upper vertebral body, a surface shape which is at least partially formed by a surface of rotation that substantially matches the shape of the milling tool. In a preferred embodiment of the invention, the axis of rotation of the milling tool is fixed on the base plate. This produces, in the underside of the upper vertebral body, a surface of rotation which is for example dome-shaped or conical and which substantially matches the top outer face of the prosthesis. In another embodiment of the invention, the axis of rotation of the milling tool can be displaceable along a transverse direction, that is to say in the frontal plane, in order to mill an oval shape, elongate in the transverse direction, in the vertebral surface.

The axis of the milling tool does not have to be perpendicular to the base plate. Instead, provision can also be made for an arrangement in which the milling tool or shaft parts arranged on the milling tool with an axis of rotation extending in the AP direction (AP=anteroposterior) can roll on the base plate or on parts of the base plate.

The invention is explained in more detail below with reference to the drawing which depicts advantageous illustrative embodiments and in which:

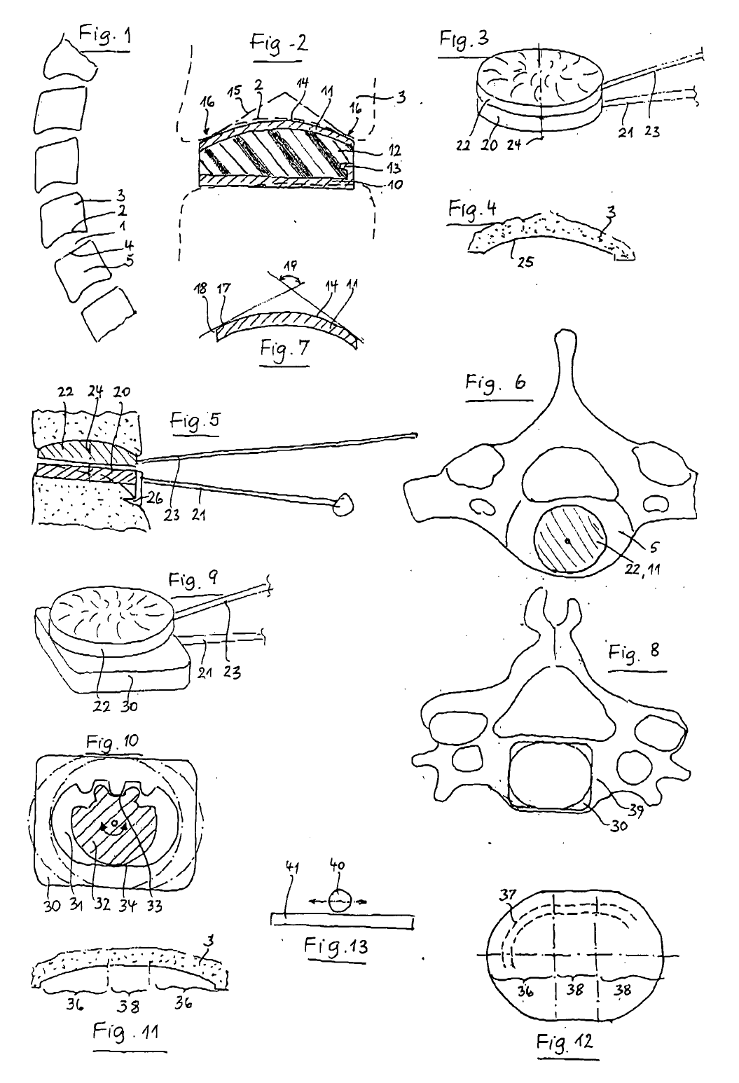

FIG. 1 shows an outline view of the cervical vertebrae in sagittal section,

FIG. 2 shows a cross section, in the medial plane, through an intervertebral prosthesis designed according to the invention,

FIG. 3 shows a milling tool for shaping the bottom surface of the upper vertebral body,

FIG. 4 shows the contour of the surface configuration produced with this tool,

FIG. 5 shows a modification of the tool according to FIG. 4,

FIG. 6 shows the prosthesis in the intervertebral space, in a plan view looking at the lower vertebra,

FIG. 7 shows the tangent angles on the top surface of the prosthesis,

FIG. 8 shows a view, corresponding to FIG. 7, with a prosthesis which is made elongate in the transverse direction,

FIG. 9 shows a milling tool for the prosthesis according to FIG. 9,

FIG. 10 shows a cross section through the tool according to FIG. 10,

FIG. 11 shows a frontal section through the bone surface shape produced with the tool according to FIG. 11,

FIG. 12 shows a plan view looking at the top surface shape produced with the tool according to FIG. 11,

FIG. 13 shows a diagrammatic illustration of an instrument with a milling tool which rolls on a base plate.

A side-on X-ray image of the cervical spine shows the contours of the vertebral bodies as illustrated in FIG. 1. It is clear from this that the intervertebral spaces 1 in sagittal section are delimited at the top by a concave bottom surface 2 of the upper vertebral body 3 and at the bottom by an approximately flat top surface 4 of the lower vertebral body 5.

From this, the invention derives the general rule that an intervertebral prosthesis should be convex at the top and made flat at the bottom.

The illustrative embodiment shown in FIG. 2 consists of a lower cover plate 10, an upper cover plate 11, and a prosthesis core 12. The cover plate 10 has a substantially flat surface extent and has, at the margin, retainer profiles 13 for retaining the prosthesis core 12. In sagittal section, the upper cover plate 11 is delimited by an outer surface contoured as a convex arc of a circle. In a known manner, the inner surface forms, together with the associated surface of the prosthesis core, a spherical sliding hinge. The inner and outer surfaces of the upper end plate 11 expediently extend parallel, that is to say concentrically, with respect to one another. The cover plates 10, 11 preferably consist of rigid material, such as metal. The prosthesis core 12 consists of a material that promotes sliding, in particular ultra-high molecular weight polyethylene.

The outer surfaces of the prosthesis cover plates are expediently designed in such a way that they fix on the bone in a manner secure against displacement. Suitable for this purpose is, for example, a rough, porous surface into whose interstices the bone substance can grow, or a surface provided with a toothed profile. Moreover, the outer surface of the cover plates 10, 11 can be equipped with a coating which promotes the connection to the bone, for example calcium hydroxyapatite.

The convex dome shape of the upper cover plate 11 has the advantage that it is entirely or partially similar to the natural concave shape of the mating bone surface. If a congruent seat surface is milled in this mating bone surface, there is therefore the chance of being able to remove less bone than is the case when using differently design d prostheses. The same applies to the lower cover plate whose flat configuration approximates to the natural shape of the mating bone surface. This can be seen clearly from FIG. 2, in which the mating bone surfaces are indicated with broken lines.

The convex shape, according to the invention, of the upper cover plate 11 of the prosthesis also has the advantage that, by means of the form fit between cover plate and bone, the prosthesis is better retained in the intervertebral space. There is practically no chance of the prosthesis being able to shift in the ventral or dorsal direction relative to the upper vertebral body 3. To achieve this goal, the outer surface of the upper cover plate 11 does not have to be spherical, although this does have the advantage that the volume of bone substance to be removed is particularly small. Instead, the surface 14 can also have a different convex shape. For example, it can have a conical design, as is indicated by dot-dash lines at 15. In any event, it is preferable that the outer surface of the upper cover plate 11 lies between the spherical surface 14 and the conical surface 15, because then there is the greatest probability that very little bone substance will have to be removed from the areas 16 lying radially to the outside, and therefore a chance that some of the resistant cortical bone will be preserved there. This is achieved in particular if the edge tangents, lying opposite one another in sagittal section, to the outer surface 14 of the upper cover plate 11 enclose an angle 19 with one another which is not greater than 90° and is preferably 60°. In accordance with FIG. 7, edge tangent is to be understood as a tangent which, in sagittal section, is located at a point 17 which is not more than 4 mm away from the edge 18 of the upper cover plate 11. If the prostheses is delimited at the top by a surface of rotation, this simplifies the operating procedure because it is not necessary to consider the orientation of the prosthesis with respect to the sagittal direction. The tool for milling the bottom surface 2 of the upper vertebral body 3 cooperating with this surface of rotation is also simplified by using the milling tool according to the invention, which is shown in FIGS. 3 and 5. It consists of a base plate 20, in which a holder part 21 is secured, and of a milling disk 22 with a handle 23. The milling disk 22 is circularly delimited and, on its top, it bears suitable milling disks. It is mounted to rotate about a center axis 24 of the base plate. In accordance with FIG. 5, the instrument can be pushed into the intervertebral space, its correct position being set by maneuvering the holder part 21 and possibly the handle 23. The instrument is then secured in the intended position by means of the holder part 21, while the handle 23 is pivoted to and fro so that the milling disk 22 is turned to and fro in order to mill the bone. The milled bone surface 25 takes on the shape of the top face of the milling disk which is identical to the shape of the outer surface 14 of the upper cover plate 11 of the prosthesis. Teeth 26 on the base plate 20 may be helpful for holding the base plate 20 stationary during the milling procedure.

After the instrument has been inserted, the distraction force is released so that the vertebral bodies 3, 5 enclosing the intervertebral space 1 at the top and bottom are pulled together by the force of the ligaments. This force generally suffices to press the milling disk 22 onto the bone during the milling procedure. The milling procedure is completed at the latest when the ligament tensioning subsides.

The height of the milling instrument (base plate 20 and milling disk 22 together) is matched to the height of the prosthesis. If, before insertion of the milling instrument, the surface 4 delimiting the intervertebral space at the bottom is made ready to receive the prosthesis, it may be expedient to make the height of the milling instrument the same as the height of the prosthesis. When, during the milling procedure, the ligament tensioning subsides, the shaped intervertebral space then has exactly the height necessary for receiving the prosthesis. The height of the milling instrument can also be kept slightly smaller than the height of the prosthesis if, after insertion of the prosthesis, a predetermined ligament tensioning is intended to exist between the vertebral bodies 3 and 5 enclosing the prosthesis.

During the milling procedure, the milling instrument has the position indicated in FIG. 6 relative to the lower vertebral body 5. This same position is adopted by the prosthesis after its insertion. In this illustration, it is assumed that the prosthesis is on the whole circular. This applies generally to the upper cover plate 11. The base plate 20 of the milling instrument does not need to have such a circular contour as the milling disk 22. On the contrary, it may be expedient for the base plate 20 to be made elongate in the lateral direction, as is shown in FIG. 9, where the base plate 30 is shown as being rectangular, with a larger dimension in the lateral direction. Precise positioning of the instrument in the intervertebral space can be made easier by such a shape, because the intervertebral space also often has a greater dimension in the lateral direction than in the AP direction.

If the intervertebral space is elongate in the lateral direction, it may be desirable to use a prosthesis which likewise has a greater dimension in the lateral direction than in the AP direction (AP=anteroposterior). In these cases, it is possible to use the illustrative embodiment described with reference to FIGS. 8 through 12. In this case it may be desirable to make not just the dimension of the lower end plate 30, but also that of the upper cover plate 11, greater in the lateral direction than in the AP direction, because in this way the loading of the bone at the boundary surface to the prosthesis can be reduced. In this case, if the shaping of the bone surface 2 intended to cooperate with the upper cover plate 11 is to be performed just as easily as was described above with reference to FIGS. 3 through 6, this instrument is modified in the manner shown in FIG. 10.

The base plate 30, which is shown as being rectangular, but which can have any contour shape that appears expedient as regards the cooperation with the associated bone surface 4, comprises a laterally elongated cutout 31 which receives a pin 32 which is connected rigidly and centrally to the milling disk 22. At the rear (or front) edge, the cutout 31 and the pin 32 have an interacting toothing 33. The front (or rear) edges 34 are smooth and designed for sliding. when the handle 23 of the milling disk 22 is pivoted to and fro relative to the holder part 21, as has been explained above, the pin 32 executes a rotation movement in the sense of the arrow indicated. Through the cooperation between the teeth 33, it is at the same time also moved to and fro in translation in the cutout 31 in the lateral direction. This same translation movement is also executed by the milling disk, so that the bone surface shaped by it assumes the shape shown in FIGS. 11 and 12. This shape is composed of two surfaces of half rotation 36 with generatrices 37 extending in arcs of a circle, and of a central surface 38 which connects them and in which the generatrices extend rectilinearly. The outer surface 14 of the upper cover plate 11 of the prosthesis also has the same shape.

Instead of arranging the milling instrument 40 with an axis extending perpendicular to the base plate 41, it can also be arranged on the latter with a parallel axis extending in the AP direction, as is shown in FIG. 13. When it is turned to and fro by means of a handle (not shown), it rolls on the base plate 41 and in so doing executes a translation movement in the arrow direction. To ensure that it does not slip relative to the base plate 41, an interacting toothing (not shown) can be provided on the milling tool 40 and the base plate 41.

1. A cervical prosthesis, comprising a lower cover plate configured to be connected to a lower vertebral body, an upper cover plate configured to be connected to an upper vertebral body, and a prosthesis core forming a connection between the upper cover plate and the lower cover plate,
wherein the lower cover plate has a substantially flat bottom surface, the upper cover plate has a top face which is convex at least in sagittal section.

2. The prosthesis as claimed in claim 1, wherein the top surface of the upper cover plate has a bulge which, in a sagittal section, lies between a circle contour with a radius of curvature of not more than 25 mm and an acute-angled contour with an apex angle of not more than 90°.

3. The prosthesis as claimed in claim 2, wherein the top surface of the upper cover plate is formed by a surface of rotation.

4. The prosthesis as claimed in claim 2, wherein the top surface of the upper cover plate is elongate in a lateral direction.

5. The prosthesis as claimed in claim 4, wherein the top surface of the upper cover plate comprises three surface portions, two outer portions of which are opposite surfaces of half rotation, a portion lying between the outer portions consisting of parallel generatrices which connect mutually facing limits of the surfaces of half rotation to one another.

6. An instrument for milling a vertebral surface cooperating with the top surface of the upper cover plate of the prosthesis as claimed in one of claims 1 through 5, comprising a base plate configured for bearing on the lower vertebral body and a milling tool mounted on the base plate for the upper vertebral body.

7. The instrument as claimed in claim 6, wherein the base plate has a contour shape adapted to the shape of the end plate of the lower vertebral body.

8. The instrument as claimed in claim 6, wherein the milling tool has an axis of rotation arranged transversely with respect to the base plate and has a working surface that substantially matches a part of the top surface of the upper cover plate.

9. The instrument as claimed in claim 8, wherein the axis of rotation of the milling tool is fixed on the base plate.

10. The instrument as claimed in claim 8, wherein the axis of rotation of the milling tool is displaceable along the base plate.

11. The instrument as claimed in claim 6, wherein the milling tool rolls on the base plate.

12. The prosthesis as claimed in claim 1, wherein the prosthesis core forms a hinged connection between the upper cover plate and the lower cover plate.

13. The instrument as claimed in claim 7, wherein the milling tool has an axis of rotation arranged transversely with respect to the base plate and has a working surface that substantially matches a part of the top surface of the upper cover plate.

14. The instrument as claimed in claim 13, wherein the axis of rotation of the milling tool is fixed on the base plate.

15. The instrument as claimed in claim 13, wherein the axis of rotation of the milling tool is displaceable along the base plate.
